(19) United States
(12) Patent Application Publication
(10) Pub. No.: US 2015/0201663 A1
(43) Pub. Date: Jul. 23, 2015

(54) PROTEIN HYDROLYSATE

(71) Applicant: DSM IP ASSETS B.V., Heerlen (NL)

(72) Inventors: Andre Leonardus De Roos, Echt (NL); Jing Shi, Echt (NL); Luppo Edens, Echt (NL)

(21) Appl. No.: 14/420,757

(22) PCT Filed: Aug. 13, 2013
(86) PCT No.: PCT/EP2013/066899

§ 371 Date: Feb. 10, 2015

(30) Foreign Application Priority Data
Aug. 14, 2012 (EP) 12180389.4

(51) Int. Cl. A23L 1/305; C07K 14/805; A23K 1/00

(52) U.S. Cl. CPC A23L 1/3053; A23K 1/007; C07K 14/805

(57) ABSTRACT

The present invention relates to a protein hydrolysate, wherein the hydrolysate has a percentage of peptides having a histidine residue at the C-terminal end of at least 10 wt % of the total peptide content of the protein hydrolysate and a process for preparing the protein hydrolysate. The invention further relates to a food or feed product comprising the protein hydrolysate.

Enzyme protein hydrolysates are widely used in the food industry and can be produced from various protein sources for instance from milk protein or soy protein (see for instance WO2002/45524 and WO2008/131008).

Another protein source from which protein hydrolysates may be derived is blood. Blood harbours valuable proteins, in particular haemoglobin. Haemoglobin consists of a globin fraction linked to an iron-binding haem group. Despite the abundance of haemoglobin derived from slaughter houses, the use of haemoglobin is limited in food processing. The main reason for this is that the strong colour, and unpleasant odour and taste are undesirable in food stuffs. Considering the continuous interest in cheap, food grade protein, it would be beneficial and desirable to utilize and expand the yield of food grade proteins from slaughter side streams. Therefore, any improvement in terms of decouloring, functionality, yield and taste of decolourized haemoglobin is considered as highly advantageous. Nowadays most of the blood derived side streams from animal slaughtering are disposed of and are seen as an environmental burden.

In the past, several methods have been described to decolourize the haemoglobin fraction of blood. Among them are methods based on chemical treatments with acetone, alcohols and acids. Also bleaching by use of peroxides has been proposed. All of these chemical approaches are disputed because of their environmental aspects, their food grade character, their effects on amino acid modification and the possibilities for forming toxic compounds.

An alternative method for decolouring haemoglobin is by enzymatic removal of the haem group (see for instance U.S. Pat. No. 4,262,022 and I. Aubes-Dufau & D. Combes, Appl. Biochem. Biotechn., 1997, Vol 67. p 127-137.

Aubes-Dufau & Combes shows the effect of different proteases, such as pepsin, alcalase and proctase on the bitterness of haemoglobin hydrolysates.

U.S. Pat. No. 4,262,022 discloses the hydrolysis of blood with the broad-spectrum protease Alcalase (subtilisin from B. licheniformis). Disadvantages of enzymatic hydrolysis of blood as disclosed in the state of the art are the risk of over-digestion of the globin moiety of haemoglobin such that a bitter tasting hydrolysate is created with limited functionality caused by the many small peptides formed.

Haemoglobin also presents a substrate that is frequently used in an assay for determining the proteolytic activity of acid proteases. In this so called HUT assay, an acid protease is incubated with denatured haemoglobin and after incubation, large remaining peptides are precipitated with tri-chloric acid, The proteolytic activity is then established by measuring the absorbance of the small peptides present in supernatant. Chang et. al. (J. Biochem 80, 975-891 (1976) and Chang & Takahashi (L. Biochem 74, 231-237 (1973) have applied this HUT test to measure the activity of acid proteases Type A and B from A. niger, but do not disclose the use of these acid proteases in the preparation of protein hydrolysates for use in food or feed.

US2005/0123932 A1 relates to nucleic acid chelating agent conjungates and discloses a peptide library comprising polyhistidine-containing recombinant peptides. US2005/0123932 does not relate to protein hydrolysates for use in food applications.

The present invention relates to an improved process for preparing a protein hydrolysate from blood which solves the problems outlined above.

### SUMMARY OF THE INVENTION

The present disclosure relates to a protein hydrolysate that has a percentage of peptides having a histidine residue at the C-terminal end of at least 10% of the total amount of peptides in the protein hydrolysate, wherein the peptides do not comprise a histidine residue at the penultimate position at the C-terminal end. The present disclosure also relates to a process for preparing a protein hydrolysate and a food product comprising the protein hydrolysate.

Advantages of the protein hydrolysate as disclosed herein are that the protein hydrolysate is colourless, does not exhibit a bitter taste, nor an off-taste related to iron in case the protein hydrolysate is derived from haemoglobin and has good functional properties, such as water-binding capacity, gelling and emulsification properties.

### DETAILED DESCRIPTION

A protein hydrolysate as disclosed herein advantageously has a percentage of peptides having a histidine residue at the C-terminal end of at least 10%, for instance at least 15%, 20%, for instance at least 25% or at least 30%, or at least 35%, such as at least 40% of the total peptide content of the protein hydrolysate. As used herein the peptide content is measured with LC-MS/MS.

A protein hydrolysate as disclosed herein has a histidine C-terminal normalized to the abundance of the histidine present in the protein hydrolysate of at least 4.

A protein hydrolysate as disclosed herein has a degree of hydrolysis (DH) of at least 10% and usually below 25%. The degree of hydrolysis (DH) may be between 12 and 22%, or between 13 and 20%, for instance between 14 and 18%. It was advantageously found that at these DH ranges the hydrolysate was colourless in the event the protein hydrolysate was derived from haemoglobin.

In addition it was found that a protein hydrolysate having a DH range as disclosed herein advantageously exhibited good functionality, such as emulsification and gelation and did not show bitterness.

In the event the protein hydrolysate is prepared from haemoglobin, a haem fraction is also formed, which can be removed from the protein hydrolysate by precipitation at a pH of 3 to 5. The DH of a protein hydrolysate derived from haemoglobin is preferably determined after removal of the haem fraction. The colouring resulting from haem is usually detected by measuring the absorbance at 405 nm. As haem comprises iron in the so-called porphyrin, the presence of haem and iron is a protein hydrolysate derived from haemoglobin is linked. Advantageously, a protein hydrolysate obtained from haemoglobin has an iron content of less than 100 ppm, or less than 95, or 90 ppm, such as less than 85 ppm. Usually the iron content is above 5, 10, 20 or 30 ppm. Such low iron content advantageously reduces the iron taste of the protein hydrolysate and makes the protein hydrolysate suitable for application in food or feed.

A protein hydrolysate as disclosed herein may have a volume of at least 100 ml, such as at least 500 ml, or at least 1 L of at least 10, or 100 L or at least 1 cubic metre (m³).

A protein hydrolysate as disclosed herein can be in liquid form and/or in dry form, for instance a freeze dried form. A skilled person in the art knows how to prepare a protein hydrolysate in liquid and/or dry form.

The protein hydrolysate as disclosed herein does not comprise trichloric acid, such as less than 100 ppm, or less than 10 ppm or less than 1 ppm. Trichloric acid can advantageously be determined by HPLC-ESI-MS/MS as disclosed in Kim et al. (2009) Toxicology, Vol 262, No. 262, p. 230-238. A protein hydrolysate as disclosed herein may for instance be used in the preparation of a food product, for instance in animal product processing, for instance in meat and/or organ meat, such as in a process for preparing sausage or ham. Alternatively, the protein hydrolysate can be added to meat prior to freezing the meat, which may minimize water losses upon thawing the frozen meat.

Alternatively, the protein hydrolysate as disclosed herein may be used in the preparation of a feed product, such as calf feed or pet food. A protein hydrolysate may be derivable from any suitable animal or vegetable derived protein source. An animal derived protein source may be blood, such as haemoglobin, or milk proteins, such as casein.

The present disclosure also relates to a process for preparing a protein hydrolysate characterized in that a protein source is incubated with a histidine specific endoprotease, and preparing the protein hydrolysate. We found that by incubating a protein source with the histidine-specific protease according to the present invention, a protein hydrolysate can be produced which did not show bitterness. In the event the protein source is haemoglobin, it was found that it was possible to selectively remove the haem fraction from haemoglobin hydrolysate, for instance by precipitation at a pH of between 3 and 5.

Incubating a protein source with a histidine-specific endoprotease in a process for preparing a protein hydrolysate as disclosed herein may be performed at a pH between 1 and 6, for instance between pH 2 and 5, for instance between pH 2.5 and 4.5. In the event the protein source in a process according to the present invention comprises haemoglobin, the process may comprise a step of incubating the haemoglobin at the pH ranges disclosed above and a step of precipitating a haem fraction at a pH of between 3 and 5, such as between 3.5 and 4.5.

Any suitable amount of protein source can be used in a process for producing a protein hydrolysate disclosed herein. A suitable amount of protein source may be between 3 to 15% wt/v of protein, or as an amount of between 4 to 12% wt/v, or an amount of between 5 to 10% wt/v of protein.

A suitable temperature at which a protein source can be incubated with the protease in a process according to the present invention may be between 10 and 60 degrees Celsius, for instance between 20 and 55 degrees Celsius, for instance between 30 and 50 degrees Celsius.

A histidine specific endoprotease in a process as disclosed herein may be present in pure form. A pure form of histidine-specific endoprotease as used herein is a histidine-specific protease, or a preparation comprising a histidine-specific protease, wherein at least 40%, 50%, 60%, 70%, 80%, 90%, 95% or more of the protease activity is derived from the histidine specific protease, wherein protease activity is expressed in HPU as defined in the method disclosed herein. The biochemical purification of aspergillopepsin II as well as determination of the cleavage specificity of this enzyme is for example disclosed in Handbook of Proteolytic Enzymes, A. J. Barrett, N. D. Rawlings and J. F. Woessner eds; Academic Press). Surprisingly, we found that pure aspergillopepsin II can be used to prepare a protein hydrolysate as disclosed herein by exploiting the hydrolytic selectivity of aspergillopepsin II for cleaving C-terminal of histidine residues.

A histidine-specific endoprotease may be obtained from any suitable microbial origin, for instance fungal origin. A histidine-specific endoprotease may be obtained from Aspergillus sp, such as Aspergillus niger. A histidine-specific protease may be an aspartic endoprotease, the term aspergillopepsin II referring to enzyme classification EC 3.4.23.19. More recently, aspergillopepsin II is also referred to as aspergilloglutamic peptidase which is classified into the peptidase family G1 (A. J. Barrett, N. D. Rawlings and J. F. Woessner Eds, 2004, Handbook of Proteolytic Enzymes, Second edition, Academic Press).

A histidine-specific endoprotease used in a process disclosed herein may have at least 70% identity to the amino acid sequence of SEQ ID NO: 1. For instance, a histidine-specific endoprotease may have at least 80, 85, 90, 95, 98, 99% identity to the amino acid sequence of SEQ ID NO:1. A histidine specific endoprotease in a process as disclosed herein may comprise SEQ ID NO: 1.

Although incubating a protein source with a histidine specific endoprotease in a process as disclosed herein results in most cases in a protein hydrolysate with no or very low bitterness, the process for preparing a protein hydrolysate may comprise adding one or more additional exoproteases or aminopeptidases, for further reducing a very low bitter note that may still be present. A suitable exoprotease may be a carboxypeptidase, for instance carboxypeptidase CPG (DSM Food Specialities, Delft, The Netherlands). A suitable aminopeptidase is for instance Corolase LAP (AB Enzymes, Darmstadt, Germany). Preferably, the additional proteases are active a similar pH and temperature as the histidine specific endoprotease.

A protein source in a process according to the present invention may be any suitable animal-derived or vegetable protein, for instance the protein source comprises haemoglobin. Haemoglobin may be used in native or in denatured form and may comprise some other fractions of whole blood in a process for preparing a protein hydrolysate as disclosed herein. Fresh blood or defrosted blood may be a suitable protein source in a process as disclosed herein. Haemoglobin may be denatured by known methods in the art for instance via acid or alkaline treatment.

Haemoglobin may be used as such, for example as obtained from lysed red blood cells, or as part of whole blood or part of blood derived product. Haemoglobin may be derived from any type of animal blood, for instance pig, cow, horse, sheep, goat, chicken or more, for instance produced in slaughterhouses. Also blood from humans, processed in clinical blood donation centers may serve this purpose.

A protein source in a process as disclosed herein may also be whole blood. Whole blood comprises plasma, white blood cells and platelets and red blood cells.

A process for preparing a protein hydrolysate as disclosed herein may further comprise a step of isolating red blood cells, lysing red blood cells, e.g. in the presence of water and recovering haemoglobin.

A process for preparing a protein hydrolysate, for instance comprising hydrolysing haemoglobin with an endoprotease as disclosed herein, may comprise a further step of separating a haem-rich fraction from the protein hydrolysate. Separating a haem-rich fraction from a protein hydrolysate fraction may be carried out via methods known to a skilled person in the art, for instance by adjusting the pH of the hydrolysate to a value between 3 and 5 followed by centrifugation or filtration. The iron-containing haem-fraction may be used as a feed additive, a natural colouring agent, or for treating anaemia.

A desirable functional property of a protein hydrolysate disclosed herein may relate to water binding, gelling properties and/or emulsifying properties. Good water binding, good gelling or good emulsifying properties can be established by methods known in the art. An example of a method that can be used for establishing emulsifying properties is illustrated in Example 5.

A process for preparing a protein hydrolysate, may further comprise a step of inactivating the histidine specific endoprotease. Inactivation of the histidine specific endoprotease may be performed by adjusting the pH of the hydrolysate to a value above 6.0 or by subjecting the hydrolysate to a heat treatment. For instance, the hydrolysate may be brought to a temperature of between 60 and 90 degrees Celsius, or between 70 and 80 degrees Celsius, at a period of between 10 sec to 15 minutes, such as between 30 sec and 10 minutes, or between 1 min and 5 min. The enzyme may also be inactivated by adjusting the temperature of the hydrolysate to between −1 and +2 degrees Celsius, for instance between 0 and 1 degrees Celsius.

The present disclosure also relates to a process for preparing a food or feed product, comprising adding a protein hydrolysate according to the present disclosure to the food or feed product or an intermediate form of the food or feed product and preparing the food or feed product. Any suitable food product may be prepared, for instance meat derived product such as ham or sausage. An intermediate form of a food product is any suitable form of a food product during its preparation. The preparation of a food product such as a sausage or a ham are known methods to a skilled person in the art.

A feed product is a product to feed animals, and may be any product known to a skilled person in the art, for instance calf feed or pet food.

Adding a protein hydrolysate in a method as disclosed herein may be performed by mixing or stirring.

Hence, the present disclosure also relates to a food or feed product comprising a protein hydrolysate as disclosed herein.

The present disclosure also relates to a packaging comprising a protein hydrolysate as disclosed herein. A suitable packaging for packaging the protein hydrolysate as disclosed herein may be a bottle, a can, a drum or a big bag.

### DEFINITIONS

Blood is a body fluid essentially composed of red blood cells (also called erythrocytes), white blood cells, platelets and blood plasma.

Haemoglobin, also spelled as hemoglobin and abbreviated Hb or Hgb, is the iron-containing oxygen-transport metallo-protein in red blood cells. Haemoglobin consists of four globular protein subunits, each connecting a non-proteinaceous porphyrin-haem group. Histidine residues play an important role in the attachment of the globin subunits to the haem group.

Haem is a prosthetic group that consists of an iron atom contained in the center of a large heterocyclic organic ring called a porphyrin.

The degree of hydrolysis (DH) is defined as the percentage of hydrolyzed peptide bonds of total peptide bonds present according to the method disclosed in Nielsen, P. M.; Petersen, D.; Dambmann, C. Improved method for determining food protein degree of hydrolysis. Journal of Food Science 2001, 66, 642-646.

The term histidine-specific refers to a preference of the enzyme to cleave peptide bonds involving a histidine residue. Determination of the preference of an enzyme to cleave peptide bonds involving a C-terminal histidine residue is carried out by LC-MC/MC using a Edans Dabcyl substrate, such as disclosed in the Materials and Methods section.

A protein hydrolysate is defined herein as a mixture of peptides and preferably low levels of free amino acids, such as below 500 micromole/g, such as below, 400, 300, 200, or 100 micromole/g, prepared by splitting a protein with an enzyme, alkali or acid.

A peptide or oligopeptide is defined herein as a chain of at least two amino acids that are linked through peptide bonds.

A polypeptide is a chain containing at least 30 amino acid residues.

A protein consists of one or more polypeptides folded into a globular or fibrous form.

The internationally recognized schemes for the classification and nomenclature of enzymes from IUMB include proteases. The updated IUMB text for protease EC numbers can be found at the internet site: http://www.chem.qmw/ac.uk/iubmb/enzvme/EC3/4/11/. The system categorises the proteases into endo- and exoproteases. An endoprotease is defined herein as an enzyme that hydrolyses peptide bonds in a polypeptide in an endo-fashion and belongs to the group EC 3.4. The endoproteases are divided into sub-subclasses on the basis of catalytic mechanism. There are sub-subclasses of serine endoproteases (EC 3.4.21), cysteine endoproteases (EC 3.4.22), aspartic endoproteases (EC 3.4.23), metalloendoproteases (EC 3.4.24) and threonine endoproteases (EC 3.4.25). Exoproteases are defined herein as enzymes that hydrolyze peptide bonds adjacent to a terminal α-amino group (“aminopeptidases”), or a peptide bond between the terminal carboxyl group and the penultimate amino acid (“carboxypeptidases”).

Sequence identity is herein defined as a relationship between two or more amino acid (polypeptide or protein) sequences, as determined by comparing the sequences. Usually, sequence identities or similarities are compared over the whole length of the sequences compared. In the art, “identity” also means the degree of sequence relatedness between amino acid sequences, as the case may be, as determined by the match between strings of such sequences.

Methods to determine identity are designed to give the largest match between the sequences tested. Methods to determine identity and similarity are codified in publicly available computer programs. Preferred computer program methods to determine identity and similarity between two sequences include BLASTP, publicly available from NCBI and other sources (BLAST Manual, Altschul, S., et al., NCBI NLM NIH Bethesda, Md. 20894). Preferred parameters for amino acid sequences comparison using BLASTP are gap open 11.0, gap extension 1, Blosum 62 matrix.

### LEGENDS

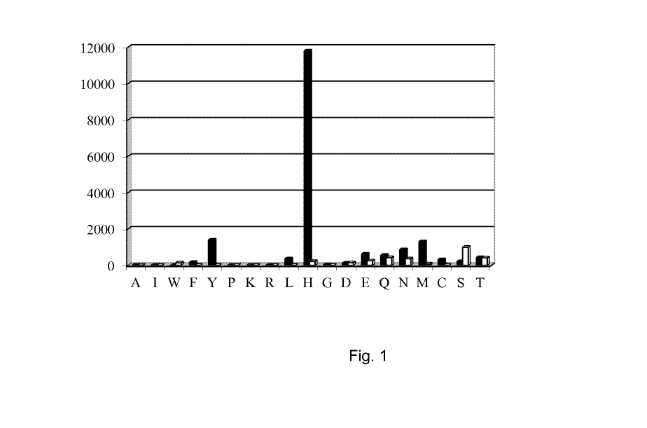

FIG. 1. Hydrolytic activity of overexpressed and chromatographically purified aspergillopepsin II towards various E(Edans)-AAXAAK-(Dabcyl)-NH₂ fluorescent substrates at pH 4 (black columns) and pH 7 (white columns). “A” in E(Edans)-AAXAAK-(Dabcyl)-NH₂ fluorescent represents alanine residues and “X” 19 different individual amino acid residues indicated by their one-letter code. At pH 4.0 (black) there exists a strong preference for cleaving peptide bonds involving histidine residues.

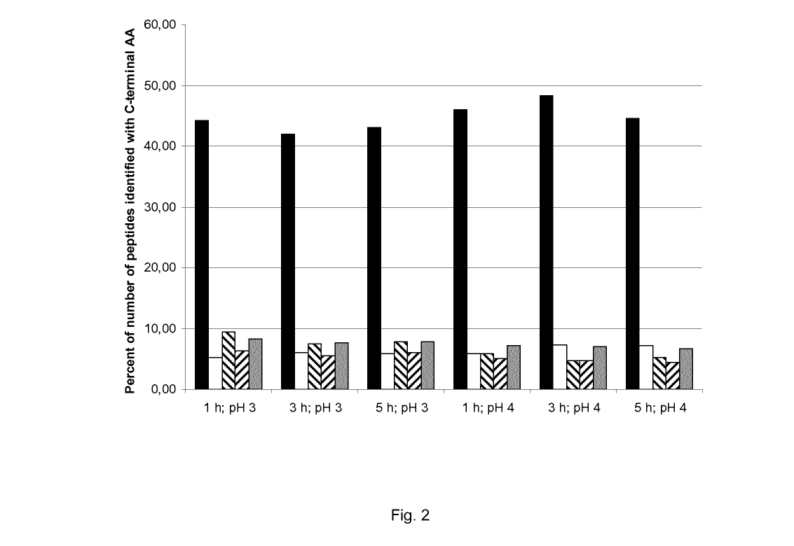

FIG. 2. Histogram showing peptides with His (H), Tyr (Y), Phe (F), Arg (R) and Asp (D) as C-terminal amino acid residue as a percentage of the total number of haemoglobin derived peptides. For each individual sample the incubation period and pH of the incubation is indicated. The bars represent the following: solid black: His (H), solid white: Tyr (Y), Downward stripes: Phe (F), Upward stripes: Arg (R), solid grey: Asp (D).

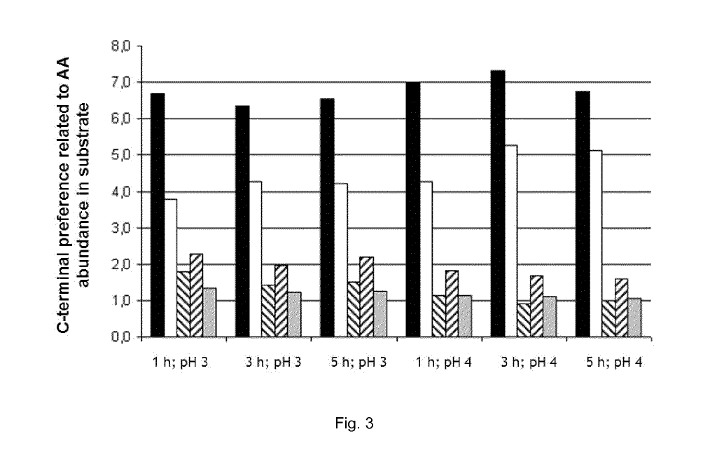

FIG. 3. Histogram showing the total number of haemoglobin peptides identified with a specified C-terminal amino acid, related to the abundance of that specific amino acid in the haemoglobin sequence. The bars represent the following: solid black: His (H), solid white: Tyr (Y), Downward stripes: Phe (F), Upward stripes: Arg (R), solid grey: Asp (D). C-terminal amino acids not mentioned represent haemoglobin peptides with a total-number-over-abundance ratio below 1.

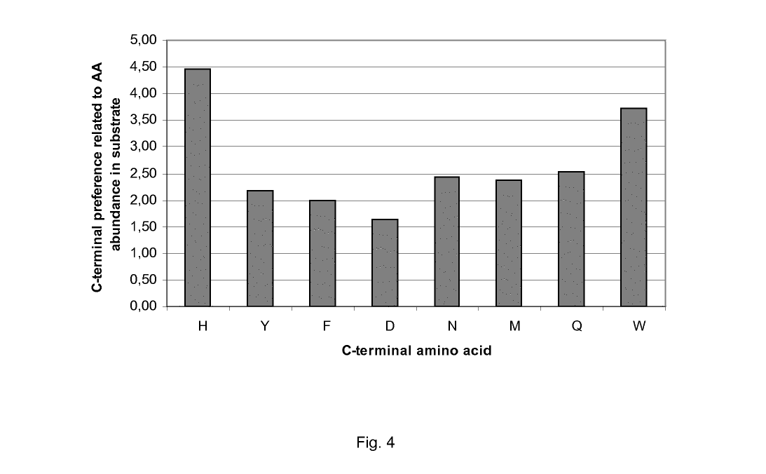

FIG. 4. Histogram showing the total number of peptides with His (H), Tyr (Y), Phe (F), Asp (D), Asn (N), Met (M), Gln (Q) and Trp (W) as C-terminal amino acid residue related to the abundance of that specific amino acid in the caseinate sequence. To achieve hydrolysis of caseinate (Alpha-S1-Casein, Alpha-S2-Casein, Beta-Casein, Kappa-Casein) the caseinate incubated with aspergillopepsin II for 3 h.

### EXAMPLES

### Materials & Methods

Haemoglobin and Hydrolysates

Commercial pig haemoglobin hydrolysate typeVepro70HLM was obtained from VEOS NV (Zwevezele, Belgium). This haemoglobin hydrolysate was obtained by treatment of haemoglobin with subtilisin. Intact red blood cells obtained by centrifugation and removing the plasma from pig blood, were also obtained from VEOS.

Production of Aspergillopepsin II

The gene for aspergillopepsin II (An01g00530; protein sequence SEQ ID NO: 1) was over-expressed in an A. niger host using methods such as described in WO 98/46772. WO 98/46772 discloses how to select for transformants on agar plates containing acetamide, and to select targeted multicopy integrants. A. niger transformants containing multiple copies of the expression cassette were selected for further generation of sample material. The transformed A. niger strain was fermented in a modified CSM-fermentation medium, pH 6.2 (40 g/l Maltose, 30 g/l Bacto-soytone, 70 g/l Sodium citrate tribasic dihydrate, 15 g/l (NH₄)₂SO₄ 1 g/l NaH₂PO₄*2H₂O, 1 g/l MgSO₄*7H₂O, 1 g/l L-Arg, 0.25 ml/l Clerol Antifoam). The culture broth obtained was filtered, sterile filtered and then concentrated by ultrafiltration. Chromatography was carried out by applying the enzyme to a Q-sepharose FF XK 26/20 column in 50 mmol/l Na-acetate, pH 5.6, followed by elution with a salt gradient. The presence of the aspergillopepsin II protein in the various fractions was quantified by judging the intensity of coloured protein bands after 4-12% SDS-PAGE (NuPAGE Bis-Tris Gel, Invitrogen).

Determination of Aspergillopepsin II Protease Activity (HPU)

20.0 g haemoglobin from bovine blood (Sigma product H2625) was suspended in approximately 700 mL water by stirring for 10 minutes at room temperature. After the addition of 3.73 g potassium chloride (KCl) the pH was adjusted to 1.75 with 0.5 mol/L hydrochloric acid. The volume of the haemoglobin suspension was adjusted to 1 L with water. The pH was checked again and adjusted to pH 1.75.

Enzyme solutions were prepared by dissolving purified aspergillopepsin II produced as disclosed above in a KCl/HCl buffer containing 3.73 g/l KCl adjusted to pH 1.75 with 2.0 mol/L HCl. To test aspergillopepsin II activity, 5 ml of the haemoglobin solution was heated at 40° C. and subsequently 1 mL enzyme solution with an activity between 5 and 25 Histidine Protease Units (HPU/mL) was added to start the reaction. After 30 minutes the reaction was stopped by adding 5 mL trichloro acetic acid solution (140 g/L) to precipitate larger peptide fragments. A blank measurement was done by adding 1.0 mL enzyme sample to a mixture of 5 mL haemoglobin solution and 5 mL trichloro acetic acid solution. The tubes were incubated at 40° C. for 30 minutes to complete the precipitation. After centrifugation, the optical density of the clear supernatant containing small peptides was measured at 275 nm. The result was compared to an L-tyrosine solution of 1.1 μg/mL.

One HPU is the amount of enzyme that hydrolyzes an amount of haemoglobin per minute, giving a solution with an optical density at 275 nm equal to the optical density of a solution containing 1.10 μg L-tyrosine per mL in 0.1 mol/L HCl solution. Conditions of the test are: pH 1.75, temperature 40 degrees Celsius, haemoglobin concentration during incubation 16.7 g/L.

Activity(HPU/mL)=(ODₛₐₘₚₗₑ−OD_{blank}/S)×11/30

### Where:

ODₛₐₘₚₗₑ: Optical density of the sample filtrate (275 nm)

OD_{blank}: Optical density of the sample blank filtrate (275 nm)

S: OD of a L-tyrosine standard solution of 1.1 μg/mL (mL/μg)

30: incubation time (minutes)

11: total volume reaction mixture (mL)

### LC-MS/MS (Liquid Chromatography-Mass Spectrometry) Analysis

Frozen protein hydrolysate samples prepared according to the procedures described in Examples 3 and 6, were thawed in a cold water bath and subsequently diluted to a protein concentration of 0.2 mg/ml by adding 0.1% formic acid (Merck, Germany). The samples were directly analysed in LC-MS system.

The hydrolysates were analysed on an Accela UHPLC (Thermo Electron, Breda, The Netherlands) coupled to a LTQ-Orbitrap Fourier Transform Mass Spectrometer (Thermo Electron, Bremen, Germany). The chromatographic separation was achieved with a 2.1×100 mm 1.8 μm particle size, 80 Å pore size, C-18 Eclipse XDB Zorbax column (Agilent Santa Clara, Calif., USA), using a gradient elution with (A) LC-MC grade water containing 0.1% formic acid B) LC-MS grade acetonitrile containing 0.1% formic acid solution (Biosolve BV, the Netherlands) as mobile phases. The 100 min gradient started from 3% Blinearly increasing to 40% B in 80 min, then washing with 80% B over 8 min and re-equilibrating with 3% B for 12 min. The flow rate was kept at 0.3 ml/min, using an injection volume of 12.5 μl and the column temperature was set to 50° C.

The mass spectrometry data acquisition was accomplished with Top 5 data-dependent acquisition using “Chromatography” and “Dynamic exclusion” options and charge states 2 and 3 included only. Resolution for the FT MS scan was 7500 and scanned for m/z range 300-2000, whereas the MS/MS experiments were performed in the ion trap. The isolation width was set at 3.0, and the normalised collision energy was set to 35.

Sorcerer (Sorcerer Software 4.0.4 build) database searching was performed with “no enzyme” option, whereas differential modification parameters oxidation (Met) and deamidation (N) were selected, with TPP (Trans-Proteomic Pipeline 4.0.2) option. The used database was an Uniprot extraction of “Sus scrofa”—proteins, which includes 1388 protein sequences. The data-base search results were filtered with protein probability of 0.95 or higher.

To assess the abundant spectral information, all mass spectra from a chromatographic run were summed up into one spectrum, which was then deconvoluted according to Thermo Scientific software. The deconvolution transforms all the multiple charged ion species into single charged species. In this way, the sum of all ion species (charge states) in the chromatogram were deconvoluted into the single charged ion species, and the most intense signals estimated. The most intense masses (defined by ion intensity above 10% of the intensity of the highest abundant ion) were manually assessed against in the database search identified peptide masses, confirming that no peptides were missed in the data base searching process.

### Degree of Hydrolysis

The Degree of Hydrolysis (DH) as obtained during incubation with the various proteolytic mixtures was monitored using a rapid OPA test (Nielsen, P. M.; Petersen, D.; Dambmann, C. Improved method for determining food protein degree of hydrolysis. Journal of Food Science 2001, 66, 642-646). The Kjeldahl factor used was 6.25.

### Determination of Iron Content

The iron content in the protein hydrolysate sample was determined with atomic emission spectrometry using a Varian Vista Pro Inductively Coupled Plasma Emission Spectrometer, equipped with, a.o. a Tecator 2040 digestion system connected with Tecator Autostep 2000 controller and a Metrohm Dosimat 765 equipped with 25 ml burette. Plasma flow was 15 l/min, auxiliary flow, 1.50 l/min, nebulizer flow 0.9 l/min. Reference solutions were ICP single element standard solution Scandium 1.000+/−0.002 g/l, and a custom made multi-element solution containing K, Na, S (1000 mg/l), Ca, Mg, P (400 mg/l) and Fe, Zn, Cu, Mn, Ni, Co, Pb, Cd (20 mg/l).

### Determination of Free Amino Acid Content

The content of free amino acids was determined using Ultra Pressure Liquid Chromatography (UPLC), using the AccQ•Tag Ultra method (Waters). The column used was Acquity UPLC BEH C₁₈ 1.7 μm, 2.1 mm×100 mm, P/N: 186002352. Mobile phase A: water/AccQ•Tag Eluent A (supplied by Waters) (90:10). Mobile phase B: AccQ•Tag Eluent B (supplied by Waters). The column temperature was 60 degrees Celsius and the tray temperature 20 degrees Celsius. The flow was maintained at 0.7 ml/min. MS/MS analyses were performed as described above.

### Example 1

### Specificity of Aspergillopepsin II for Cleaving Peptide Bonds Involving Histidine

The cleavage preference of aspergillopepsin II was tested using an_E(Edans)-AAXAAK-(Dabcyl)-NH₂ (SEQ ID NO: 2) fluorescent substrate kit with “A” representing alanine residues and “X” the 19 different individual amino acid residues as specified by their one-letter code. Dabcyl quenches the Edans fluorescence when the substrate is intact and no longer quenches the Edans fluorescence when the substrate is cleaved. The substrate is therefore called a Fluorescence resonance energy transfer (FRET) peptide. Substrate stock solutions were prepared in DMSO in 5 mM concentration. The reaction mixture contained: 195 microliters of either 100 mM Na-acetate buffer, pH 4.0 or 100 mM Tris-HCl buffer, pH 7.0 to which 2 microliters substrate stock solution and 5 microliters aspergillopepsin II (0.25 mg/ml in 50 mM Na-acetate, pH 5.6) were added. The reaction mixture was incubated in Tecan equipment (Mannedorf, Switzerland) at 37° C. for 60 min (λₑₓ=340 nm, λₑₘ=485 nm). The enzyme activity was determined in relative fluorescent units (rfu) per minute per mg of protein. As shown in FIG. 1, among the various E(Edans)-AAXAAK-(Dabsyl)-NH₂ (SEQ ID NO: 2) substrates tested at pH 4, substrate E(Edans)-AAHisAAK-(Dabsyl)-NH₂ (SEQ ID NO:3) yields by the highest rfu value for aspergillopepsin II. The implication of this is that apparently aspergillopepsin II has a high preference for cleaving substrate E(Edans)-AAHisAAK-(Dabsyl)-NH₂ at pH 4, that is, when the amino acid at position X is histidine.

To detect possible proteolytic side activities, the enzyme preparation was also incubated with different chromogenic pNA substrates. Substrate stock solutions for aminopeptidases (X-pNA), dipeptidyl aminopeptidases (A-X-pNA), tripeptidyl aminopeptidases (A-A-X-pNA), where “X”=“A”, “L”, “F”, “K”, “P”, “D” (all in the one-letter code for amino acids) were prepared in DMSO in 150 mmol/l concentration. Stock solutions for endoproteases with blocked N-end (Z-A-A-X-pNA, where ““X”=“A”, “L”, “G”, “I”, “V”, “S”, “P”, “Q”, “E”, “R”) were prepared in DMFA. The enzyme reactions were followed at 405 nm. Reactions were carrying out at 37° C. for 1 hour, at pH 4 and 7. Significant proteolytic side activity that could possibly interfere with the conclusion that aspergillopepsin II has a strong preference for cleaving peptide bonds involving histidine could not be identified.

### Example 2

### Determination Cleavage of Aspergillopepsin II at C- or N-Terminal of Histidine

As shown in Example 1, aspergillopepsin II from Aspergillus niger shows a high activity on the synthetic substrate E(Edans)-AAHAAK-(Dabcyl)NH₂ (SEQ IS NO:3). The alanine (A) groups are known not to be substrate for protease activity, which makes the peptide bonds involving histidine (H) the only protease cleavage side. In order to determine whether aspergillopepsin II cleaves N- or C-terminally of the H. Mass Spectrometry was performed,

The E(Edans)-AAHAAK-(Dabcyl)NH₂ substrate was dissolved in 50 mM CH₃COONH₄ at pH 4, to a final concentration of 1 mg/ml to create a stock solution. To 500 microliter stock solution a spatula tip <0.5 mg of purified aspergillopepsin II was added. The solution was incubated at 37° C. and 10 microliter aliquots were taken every 30 minutes. The 10 microliter aliquots and 10 microliter samples of the original stock were 1000× diluted in 50/50/0.1 MilliQ water/acetonitrile/formic acid. These diluted samples were measured on the QTOFII and Orbitrap, using direct infusion.

The substrate was analyzed with MS before and during incubation with the enzyme. The data obtained showed that the substrate was indeed degraded and according to the MS spectra predominantly fragments of m/z 675.3 and 539.3 are formed upon incubation. The m/z 675 fragment was shown to represent E(Edans)-AAH and the m/z 539.3 fragment AAK-(Dabcyl)NH2. The E(Edans)-AAHAAK-(Dabcyl)NH₂ starting material has M 1194.5 and shows up as M+2H+, m/z 598.3.

The results herein obtained demonstrate that aspergillopepsin II cleaves histidine at the C-terminal in the E(Edans)-AAHAAK-(Dabcyl)NH₂ substrate.

### Example 3

### Production of a Protein Hydrolysate with C-Terminal Histidine Residues from Intact Haemoglobin with Aspergillopepsin II

A haemoglobin solution was prepared by haemolysing erythrocytes derived from porcine-blood (33% dry matter) with 4.2 part of tap water. The pH of the diluted haemoglobin was adjusted with 4N sulfuric acid to a pH of either 4.0 or 3.0. Then aspergillopepsin 11 (13300 HPU/g) was added to a level of 1 wt % (gram enzyme solution/gram haemoglobin dry matter) and the haemoglobin—enzyme solution was incubated in a shaking waterbath at 55 degrees Celsius. Samples were taken at 1 h, 2 h, 3 h, 4 h and 5 h. At each time point the reaction was stopped by placing the samples in ice water, After centrifugation (10,000 rcf, 10 min, 4 degrees Celsius, Centrifuge 5417R, Eppendorf, USA), the supernatants were collected and stored at −20 degrees Celsius for further analysis.

The protein hydrolysate samples obtained were diluted to a protein concentration of 0.2 mg protein/ml by adding formic acid and directly analysed by LC-MS according to the procedure as disclosed in the Material & Methods section.

About 300-400 different peptides were identified that originated from haemoglobin alpha and beta and less than 10 peptides that originated from other proteins. Only peptides originating from haemoglobin were taken into account.

As shown in FIG. 2, the pH of the incubation (i.e. 3 or 4) or the incubation period (i.e. 1, 3 or 5 hours) with aspergillopepsin II had almost no effect on the percentage of peptides bearing a specific C-terminal amino acid (i.e. His, Tyr, Phe, Arg or Asn) and more than 40% of all identified peptides bear a C-terminal histidine in the protein hydrolysates obtained from haemoglobin. The latter result corroborates the data obtained with the synthetic E(Edans)-AAXAAK-(Dabcyl)NH₂ peptides described in Example 1.

In haemoglobin, histidine is a relatively abundant amino acid (6.6% of all amino acid residues). A random cleavage of peptide bonds in haemoglobin would therefore lead to 6.6% of the peptides having C-terminal His residue. To assess the cleavage preference and taking into account the abundance of amino acid residues in the haemoglobin alpha and beta chains, the percentage of peptides identified with a certain C-terminal amino acid was normalized to the presence of that particular amino acid in the haemoglobin sequence. According to the amino acid sequence of haemoglobin, the abundance of His is 6.6%, the abundance of Tyr is 1.4%, the abundance of Phe is 5.2%, the abundance of Arg is 2.7% and the abundance of Asp is 6.3%.

Thus, the ratio of peptides with a specific C-terminal amino acid divided by the abundance of that specific amino acid then gives the normalized preference. This normalized preference is shown in FIG. 3. As shown, the normalized preference of aspergillopepsin II for histidine (“H”) is more than 6, for Tyr (“Y”) around 4 and for Phe (“F”), Arg (“R”) and Asp (“D”) more than 1. For all the other amino acids this value is below 1.

Again these data illustrate the preference of aspergillopepsin II for cleaving peptide bonds involving histidine.

Finally this histidine preference of aspergillopepsin II was assessed by taking into account the most intense signal responses in the chromatographic runs in the LC-MS analysis and deconvoluted to one combined mass spectrum. The results from this assessment are in line with the results above, indicating that these most prominent peptides also have His at their C-terminus.

### Example 4

### Decoulorizing Haemoglobin with Aspergillopepsin II

Haemoglobin hydrolysates were prepared by adding either 1 ml or 2 ml of a 13300 HPU/g aspergillopepsin 11 solution to a solution of 100 g haemoglobin dry matter in 1600 ml of tap water. Incubation was performed at pH 4 at 55° C. and samples were taken after 1 h, 2 h, 3 h, 4 h and 5 h of incubation. The reaction was stopped by decreasing the temperature to 0° C. with ice, and after centrifugation the supernatants were collected. The colour, which is a measure for residual haem, of these supernatants was quantified by measuring the absorbance at 405 nm by using TECAN GENios Microplate Reader (Mannedorf, Switzerland). From the data shown in Table 1 it is clear that incubation of haemoglobin with aspergillopepsin II at pH4 reduces the colour of the supernatant in a dosage-dependent manner.

TABLE 1
Absorbance at 405 nm of haemoglobin solutions, that
were incubated with aspergillopepsin II at pH 4.0.

| | |
|---|---|
| Enzyme | |
| dosage | Incubation time [h] |

| [%] | 0 | 1 | 2 | 3 | 4 | 5 |
|---|---|---|---|---|---|---|
| 1 | 120 | 102.7 | 18.8 | 2.2 | 1.4 | 0.9 |
| 2 | 120 | 33.3 | 1.1 | 0.5 | 0.3 | 0.2 |

### Example 5

### Emulsifying Properties of Protein Hydrolysate Prepared with Aspergillopepsin II

The haemoglobin hydrolysate prepared with 1% aspergillopepsin II (13300 HPU/g) at pH 4 at 55° C. for 5 hours as disclosed in example 4, was freeze-dried and dissolved in tap water in a concentration of 50 g/l to test its emulsifying properties. In Table 2 the DH, iron content and free amino acid content, determined as described in the Materials and Methods section, are shown of this haemoglobin hydrolysate and the commercial haemoglobin hydrolysate VEPRO70HLM.

These data illustrate that the haemoglobin hydrolysates obtained with aspergillopepsin II have a lower iron content and less free amino acids than a commercially available enzymatic haemoglobin hydrolysate (VEPRO70HLM). Moreover, the DH of the hydrolysate according to the invention is significantly lower than the DH of the commercial preparation.

TABLE 2
Iron content, DH and free amino acid content
of freeze dried haemoglobin hydrolysates.

| | Iron content | DH | Free amino acid |
|---|---|---|---|
| Product | [ppm] | [%] | [μmol/g] |
| Intact haemoglobin | 3040 | 5 | n.a. |
| Haemoglobin hydrolysates | 48-80 | 14 | 31-58. |
| prepared by 1% aspergillopepsin | | | |
| II (13300 HPU/g) at 55° C. at | | | |
| pH 4 for 5 hours. | | | |
| VEPRO70HLM | 111 | 40. | 701 |

Sunflower oil was added to protein hydrolysate (pH 6.8) obtained and described above and VEPRO70HLM under high speed mixing (8,000 rpm, 5 min, IKA® T25 digital ULTRA-TURRAX®, Germany) into the water phase to create a protein:water:oil ratio of 1:20:20. Immediately after mixing, oil droplet sizes were measured by a Laser Diffraction Particle Size Analyzer LS 13320 (Beckman Coulter B.V. Woerden, the Netherlands) or, in case of heavy protein aggregation, by light microscopy (Olympus CX41, Japan). Then the two emulsions were stored at room temperature and after three days the droplet sizes were measured again. An identical approach as the aspergillopepsin II treated haemoglobin hydrolysate was followed for commercially available haemoglobin hydrolysate (VEPRO70HLM). The emulsion stabilization data thus obtained are shown in the Table 3.

TABLE 3
Emulsifying properties of hemoglobin hydrolysates

| | | Average droplet size |
|---|---|---|
| | Initial average droplet | [μm] after storage for |
| Hydrolysate | size [μm] | 3 days |
| Aspergillopepsin II - | 25 | 25 |
| treated haemoglobin | | |
| hydrolysate | | |
| VEPRO70HLM | Immediate phase | — |
| | separation | — |

From the data in Table 3 it can be concluded that the haemoglobin hydrolysate prepared by hydrolyzing haemoglobin with aspergillopepsin II has better emulsifying properties than the commercially available enzymatic haemoglobin hydrolysate VEPRO70HLM, because the latter showed immediate phase separation.

### Example 6

### 6.1 Decolouring Whole Blood with Aspergillopepsin II

Whole blood hydrolysates were prepared by adding either 250 microliter or 500 microliter of a 10000 HPU/g Aspergillopepsin II solution to a suspension of 100 g porcine blood (with 0.8% w/v sodium citrate as anti-clotting agent) in 400 g of tap water. Incubation was performed at pH 2.5 at 55 degrees C. and samples were taken after 0.5 hr, 1 hr, 1.5 hr and 2 hr of incubation. The reaction was stopped by decreasing the temperature to 0 degrees C. with ice. The pH of the system was then adjusted to pH 4 and the supernatants of the various hydrolysates were collected after centrifugation. The color of these supernatants was quantified by measuring the absorbance at 405 nm by using TECAN GENios Microplate Reader (Mannedorf, Switzerland). From the data shown in Table 4 it is clear that incubation of whole blood with aspergillopepsin II at pH2.5 reduces the colour of the supernatant in a dosage-dependent and incubation time-dependent manner. Therefore, aspergillopepsin II can be used to prepare almost colorless hydrolysates from whole blood.

TABLE 4
Absorbance at 405 nm of blood solutions incubated
with aspergillopepsin II at pH 2.5

| Enzyme | | | | | |
|---|---|---|---|---|---|
| dosage | Incubation time | | | | |
| [%]* | 0 h | 0.5 h | 1 h | 1.5 h | 2 h |
| 0.25% | 83 | 63 | 26 | 1 | 0.5 |
| 0.5% | 83 | 31 | 0.61 | 0.34 | 0.27 |

*Enzyme dosage % is defined as ml enzyme/gram blood

### 6.2. DH an Iron Content of Whole Blood Treated with Aspergillopepsin II an Alcalase 2.4 L

Whole blood hydrolysates were prepared by adding either 500 microliter of a 10000 HPU/g Aspergillopepsin II solution or 1500 microliter of a 2.4 AU-A/g Alcalase 2.4 L (Novozyme, Denmark) solution to a suspension of 100 g porcine blood (with 0.8% w/v sodium citrate as anti-clotting agent) in 400 g of tap water. Incubation was performed at either pH 2.5 (Aspergillopepsin II) or pH 8.5 (Alcalase 2.4 L) at 55 degrees C. and samples were taken after 0.5 hr, 1 hr, 1.5 hr and 2 hr of incubation. The reaction was stopped by decreasing the temperature to 0 degrees C. with ice. The pH of the system was then adjusted to pH 4 and the supernatants of the various hydrolysates were collected after centrifugation. Samples incubated for 1 hr had similar absorbance at 405 nm (around 1). These hydrolysates were further compared for iron content and degree of hydrolysis (DH).

From the data shown in Table 5 it is clear that at a similar Absorbance (decolouration) blood hydrolysate obtained with Aspergillopepsin II had a lower iron content than a blood hydrolysate obtained with Alcalase, while the DH of blood hydrolysates obtained with Aspergillopepsin II was significantly lower than the DH of blood hydrolysate obtained with Alcalase.

TABLE 5
Iron content and DH of whole blood hydrolysates
produced with different proteases

| | Iron content | |
|---|---|---|
| Product | [ppm] | DH [%] |
| Aspergillopepsin II-treated hydrolysates | 1.8 | 14.6 |
| Alcalase 2.4L-treated hydrolysates | 1.93 | 31 |

### Example 7

### Production of a Protein Hydrolysate with C-Terminal Histidine Residues from Whole Blood with Aspergillopepsin II

A blood solution was prepared by diluting 1 part of porcine blood with 4 part of tap water. The pH of the diluted solution was adjusted with 4N sulfuric acid to a pH of 2.5. Then aspergillopepsin II (10000 HPU/g) was added to a level of either 0.05 or 0.1 wt % (gram enzyme solution/gram diluted blood) and the blood—enzyme solution was incubated in a shaking waterbath at 55 degrees Celsius. Samples were taken at 0.5 h, 1 h, 1.5 h and 2 h. At each time point the reaction was stopped by placing the samples in ice water, After centrifugation (10,000 rcf, 10 min, 4 degrees Celsius, Centrifuge 5417R, Eppendorf, USA), the supernatants were collected and stored at −20 degrees Celsius for further analysis.

The protein hydrolysate samples obtained were diluted to a protein concentration of 0.2 mg protein/ml by adding formic acid and directly analysed by LC-MS according to the procedure as disclosed in the Material & Methods section.

More than 80% of the peptides identified in sample 2-9 (Table 6) are from haemoglobin, and the other 20% are from serum albumin, beta-globulin and theta haemoglobin. The histidine content in haemoglobin, serum albumin, beta-globulin and theta haemoglobin is 6.6%, 2.8%, 4.8% and 4.8%, respectively. A random cleavage of peptide bonds in haemoglobin would therefore lead to less than 7% of the peptides having C-terminal His residue. Table 6 illustrates again a clear preference of Aspergillopepsin II for cleaving peptide bonds next to histidine for a mixture of blood proteins.

TABLE 6
Number of unique peptides, number of peptides with His at
C-term., and % C-terminal His of whole blood hydrolysates.

| | | # unique | # Peptides C-term. | % C-terminal |
|---|---|---|---|---|
| Sample nr. | Remarks | peptides | His | His |
| 1 | whole blood, incubated at pH 2.5 | 0 | 0 | 0 |
| | for 2 hr (no enzyme) | | | |
| 2 | whole blood, 0.5% ZBJ, pH 2.5, 0.5 hr | 58 | 26 | 45 |
| 3 | whole blood, 0.5% ZBJ, pH 2.5, 1 hr | 135 | 64 | 47 |
| 4 | whole blood, 0.5% ZBJ, pH 2.5, 1.5 hr | 119 | 61 | 51 |
| 5 | whole blood, 0.5% ZBJ, pH 2.5, 2 hr | 120 | 54 | 45 |
| 6 | whole blood, 1% ZBJ, pH 2.5, 0.5 hr | 122 | 54 | 44 |
| 7 | whole blood, 1% ZBJ, pH 2.5, 1 hr | 150 | 70 | 47 |
| 8 | whole blood, 1% ZBJ, pH 2.5, 1.5 hr | 150 | 65 | 43 |
| 9 | whole blood, 1% ZBJ, pH 2.5, 2 hr | 184 | 76 | 41 |

### Example 8

### Production of Protein Hydrolysate with Aspergillopepsin II from Sodium Caseinate

Haemoglobin is exceptionally rich in histidine residues (about 6%). In this Example we show that Aspergillopepsin II can also be used to generate hydrolysates rich in peptides carrying C-terminal histidines from other protein substrates. To that end bovine caseinate, an industrially important product incorporating about 2% of histidine, was incubated with Aspergillopepsin II according to the invention, and the resulting hydrolysate was then subjected to LC/MS analysis.

A 0.5%(w/w) sodium caseinate solution was prepared by dissolving 0.15 g sodium caseinate salt (Sigma) into 29.85 g tap water. The pH of the solution was adjusted to pH 3 using 4N sulphuric acid and then 1.5 μl of a 13300 HPU/g Aspergillopepsin II solution was added to the solution. The enzymatic incubation was performed in a shaking waterbath at 55° C. The reaction was stopped after 3 h incubation by placing the sample in ice water. The sample was stored at −20° C. for further analysis.

Following the procedure as outlined in the Materials & Methods section, the enzymatically hydrolysed caseinate sample was diluted to a protein concentration of 0.2 mg protein/ml by adding formic acid and directly analysed in LC-MS system.

To assess the cleavage preference and taking into account the abundance of amino acid residues in sodium caseinate, the percentage of peptides identified with a certain C-terminal amino acid was normalized to the occurrence of that particular amino acid in the caseinate sequence. Thus, the ratio of caseinate derived peptides with a specific C-terminal amino acid divided by the abundance of that specific amino acid in the caseinate substrate then gives the normalized preference. From the normalized preference shown in FIG. 4 it is clear that the preference of aspergillopepsin II for cleaving next to histidine residues also applies to proteins with relatively low histidine contents.

The examples show that a protein hydrolysate with a high percentage of peptides with histidine at the C terminal can be produced with aspergillopepsin II. The examples further show that a protein hydrolysate derived from haemoglobin has a low iron content and good emulsifying properties.

1. A protein hydrolysate, wherein the hydrolysate has a percentage of peptides having a histidine residue at the C-terminal end of at least 10 wt % of the total amount of peptides in the protein hydrolysate, wherein the peptides do not comprise a histidine residue at the penultimate position at the C-terminal end.

2. A protein hydrolysate according to claim 1, wherein the degree of hydrolysis (DH) is between 10 and 25%, as defined as the percentage of hydrolyzed peptide bonds of total peptide bonds present.

3. A protein hydrolysate according to claim 1, wherein the hydrolysate is a haemoglobin hydrolysate.

4. A protein hydrolysate according to claim 1, wherein the hydrolysate has an iron content of less than 100 ppm.

5. A protein hydrolysate according to claim 1, wherein the hydrolysate has a content of free amino acids of less than 500 micromole/g.

6. A process for preparing a protein hydrolysate according to claim 1, comprising incubating a protein source with a histidine specific endoprotease, and preparing the protein hydrolysate.

7. A process according to claim 6, wherein the protein source comprises haemoglobin.

8. A process according to claim 6, wherein an amount of 3 to 15% wt/v of the protein source is incubated with a histidine specific endoprotease.

9. A process according to claim 6, wherein the protein source is incubated at a pH of between 1 and 6.

10. A process according to claim 6, wherein the histidine-specific endoprotease is derivable from Aspergillus sp.

11. A process according to claim 6, wherein the histidine-specific endoprotease has at least 70% identity to the amino acid sequence of SEQ ID NO: 1.

12. A process according to claim 7, comprising separating a haem fraction from the protein hydrolysate.

13. A process for preparing a food or feed product, comprising adding a protein hydrolysate according to claim 1 to the food or feed product or an intermediate form of the food or feed product and preparing the food or feed product.

14. A packaging comprising the protein hydrolysate according to claim 1.

15. A food or feed product comprising a protein hydrolysate according to claim 1.

16. A food product according to claim 15, wherein the food product is an animal-derived product.


SEQUENCE LISTING (3 sequences)

SEQ ID NO: 1 - protein, 282 aa, Aspergillus niger
MKFSTILTGSLFATAALAAPLTEKRRARKEARAAGKRHSNPPYIPGSDKEILKLNGTSNEDYSSNWAGAVLIGDGYTKVTGEFTVPSVSAGSSSSSGYGGGYGYYKNKRQSEEYCASAWVGIDGDTCETAILQTGVDFCYEDGQTSYDAWYEWYPDYAYDFNDITISEGDTIKVTVEATSKSSGSATVENLTTGQSVTHTFSGNVEGDLCETNAEWIVEDFESGDSLVAFADFGSVTFTNAEATSDGSTVGPSDATVMDIEQDGTVLTETSVSGDSVTVTYV

SEQ ID NO: 2 - protein, 6 aa, Artificial Sequence
  Xaa in the Edans-Dabcyl substrate can be any amino acid
AAXAAK

SEQ ID NO: 3 - protein, 6 aa, Artificial Sequence
  Edans-Dabcyl substrate
AAHAAK